(19) United States
(12) Patent Application Publication
(10) Pub. No.: US 2023/0038895 A1
(43) Pub. Date: Feb. 9, 2023

(54) SIGNAL TRANSFORMER ARTIFICIAL INTELLIGENCE

(71) Applicant: Intel Corporation, Santa Clara, CA (US)

(72) Inventors: Amit Bleiweiss, Yad Binyamin (IL); Gideon Klimer, Santa Clara, CA (US); Yael Malerevich, Santa Clara, CA (US); Ganit Prisant Henkin, Santa Clara, CA (US); Estelle Guez, Tel Aviv (IL)

(21) Appl. No.: 17/937,328

(22) Filed: Sep. 30, 2022

Related U.S. Application Data
(60) Provisional application No. 63/352,077, filed on Jun. 14, 2022.

(51) Int. Cl. A61B 5/00; G06T 11/60; A61B 5/339; A61B 5/308

(52) U.S. Cl. CPC A61B 5/7267; G06T 11/60; A61B 5/339; A61B 5/308

(57) ABSTRACT

Systems, apparatuses and methods may provide for technology that converts a plurality of multi-channel time-synchronized signals into a plurality of image patches, combines the plurality of image patches into an image, and generates, by a transformer neural network, a classification result based on the image.

### CROSS-REFERENCE TO RELATED APPLICATIONS

The present application claims the benefit of priority to U.S. Provisional Application No. 63/352,077 filed on Jun. 14, 2022.

### TECHNICAL FIELD

Embodiments generally relate to artificial intelligence (AI) technology. More particularly, embodiments relate to signal transformer AI technology.

### BACKGROUND OF THE DISCLOSURE

Signals in the healthcare domain (e.g., electrocardiogram/ECG signals for cardiology) may include measurements that are multi-channel in nature (e.g., taken from several leads) and time-synchronized. Although efforts may have been made to analyze ECG signals with AI technology, there remains considerable room for improvement. For example, AI attempts to treat each measurement as a one-dimensional (1D) convolution have not been successful in scaling to multiple signal inputs due to an inability to retain the expressivity of the signal. More particularly, 1D convolutions typically smooth and quantize the signal, which may cause a loss of critical anomalies. Additionally, research publications such as ECG-DualNet₂ have attempted to combine the direct signal with other data sources (e.g., images, spectrogram, etc.), but have not achieved acceptable results. Moreover, the ECG-DualNet₂ solution is not able to handle multi-channel simultaneous signals (e.g., ECG signals are typically taken from twelve leads).

### BRIEF DESCRIPTION OF THE DRAWINGS

The various advantages of the embodiments will become apparent to one skilled in the art by reading the following specification and appended claims, and by referencing the following drawings, in which:

FIG. 1 is an illustration of an example of an ECG measurement configuration and corresponding signal according to an embodiment;

FIGS. 2A-2B are illustrations of an example of an electroencephalogram (EEG) measurement configuration and corresponding signal according to an embodiment;

FIG. 3 is an illustration of an example of an electromyography (EMG) measurement configuration and corresponding signal according to an embodiment;

FIG. 4 is an illustration of an example of a cardiotocography (CTG) signal according to an embodiment;

FIG. 5 is a block diagram of an example of an automated classification architecture according to an embodiment

FIG. 6 is an illustration of an example of an explainability of an automated classification architecture according to an embodiment;

FIG. 7 is an illustration of an example of per pixel explainability images for ground truth inputs according to an embodiment;

FIG. 8 is an illustration of a set of feature images at various stages of an encoder layer according to an embodiment;

FIG. 9 is an illustration of an example of a back-projection of a heatmap onto an original ECG plot according to an embodiment;

FIG. 10 is an illustration of an example of a classification result according to an embodiment;

FIG. 11 is an illustration of an example of a raw data classification result according to an embodiment;

FIG. 12 is an illustration of an example of correlation metric classification result according to an embodiment;

FIG. 13 is a flowchart of an example of a method of classifying signals according to an embodiment;

FIG. 14 is a flowchart of an example of a method of using a video transformer neural network to classify signals according to an embodiment;

FIG. 15 is a block diagram of an example of a performance-enhanced computing system according to an embodiment;

FIG. 16 is an illustration of an example of a semiconductor package apparatus according to an embodiment;

FIG. 17 is a block diagram of an example of a processor according to an embodiment; and

FIG. 18 is a block diagram of an example of a multi-processor based computing system according to an embodiment.

### DETAILED DESCRIPTION

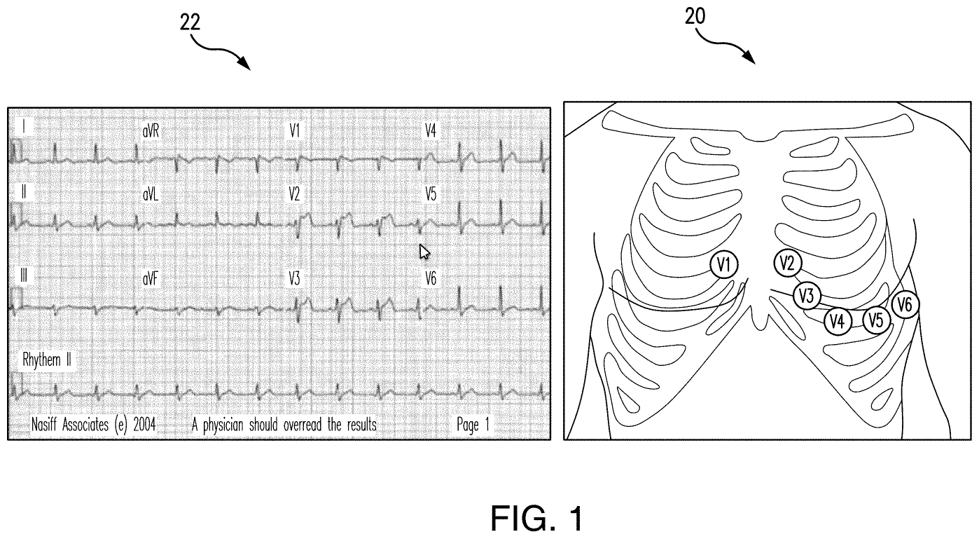

Turning now to FIG. 1 an electrocardiogram (ECG) measurement configuration (e.g., Holter monitor) is shown in which a plurality of leads (e.g., V1-V6) are used to collect cardiology-related measurements from a subject (e.g., patient, individual). In the illustrated example, the leads generate a plurality of signals 22 that are multi-channel in nature (e.g., each signal from a lead corresponds to a channel) and time-synchronized (e.g., recording both simple 5,000 time steps, and up to 24 hours). As will be discussed in greater detail, the technology described herein converts the signals 22 into a plurality of image patches, combines the image patches into an image, and generates, by a transformer neural network (e.g., deep learning model that uses self-attention to process sequential input data), a classification result based on the image. In one example, the classification result facilitates the automated detection of cardiology anomalies and/or diseases (e.g., irregular heartbeats/arrhythmias) in the subject.

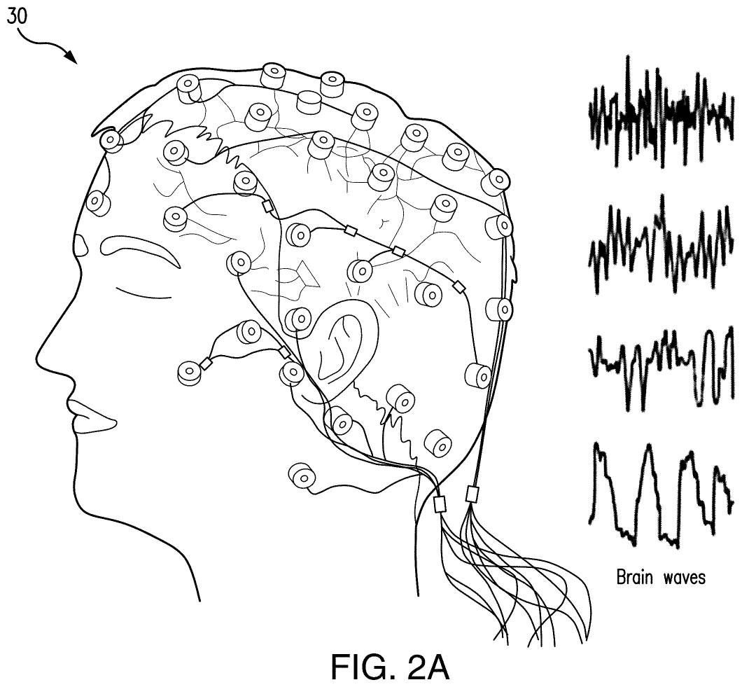

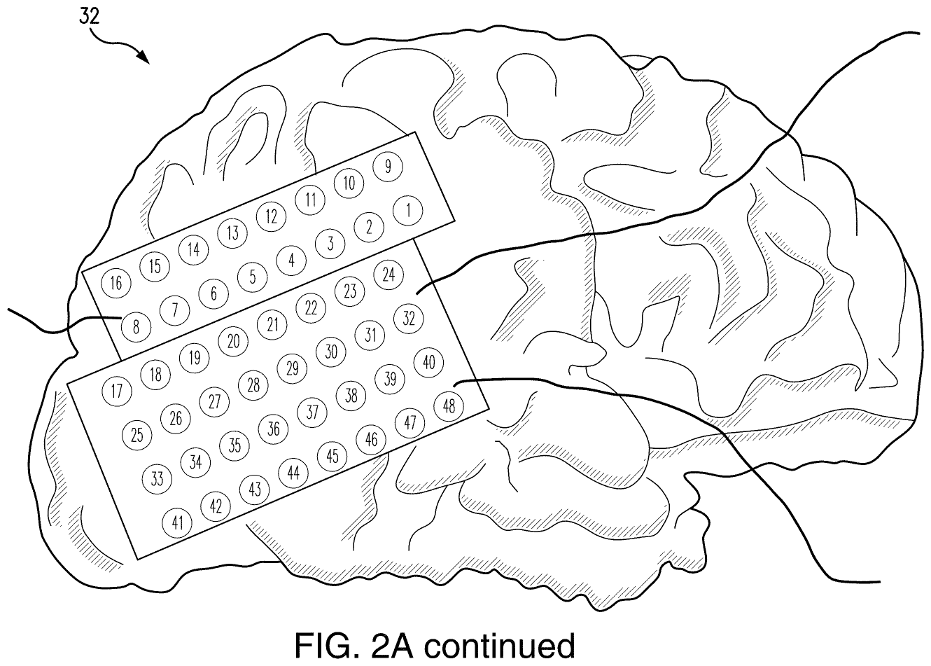

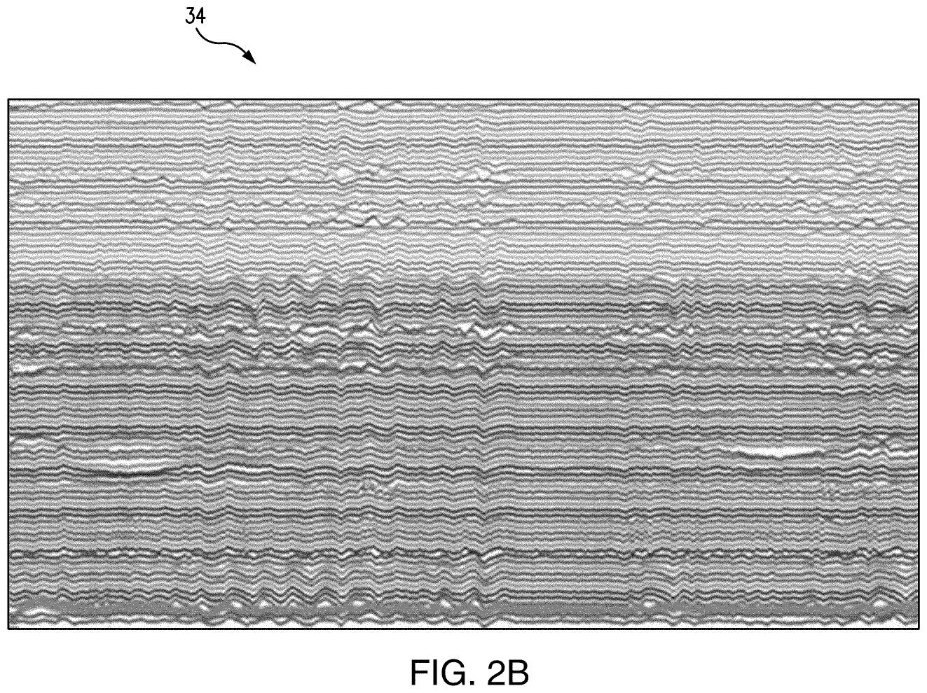

FIGS. 2A and 2B show electroencephalogram (EEG) measurement configurations 30, 32 in which a plurality of leads (e.g., leads 1-48) are used to collect neurology-related measurements (e.g., using both fast and slow frequency with a wide range of locations and applications) from a subject. In the illustrated example, the leads generate a plurality of multi-channel time-synchronized signals 34. As will be discussed in greater detail, the technology described herein converts the multi-channel time-synchronized signals 34 into a plurality of image patches, combines the image patches into an image, and generates, by a transformer neural network, a classification result based on the image. In one example, the classification result facilitates the automated detection of neurology anomalies and/or diseases (e.g., epilepsy, dementia, etc.).

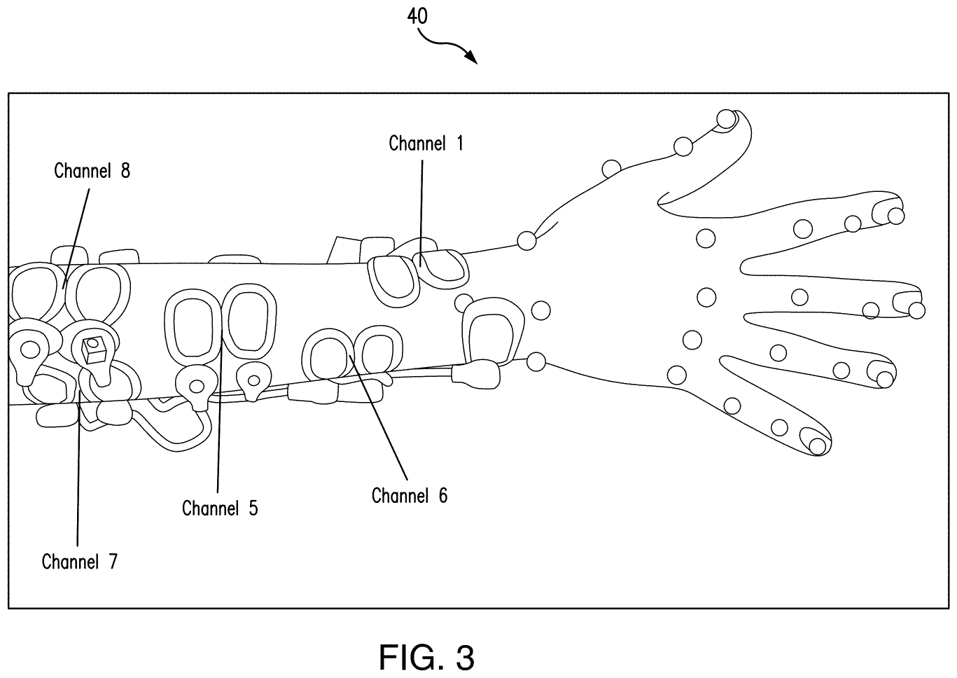

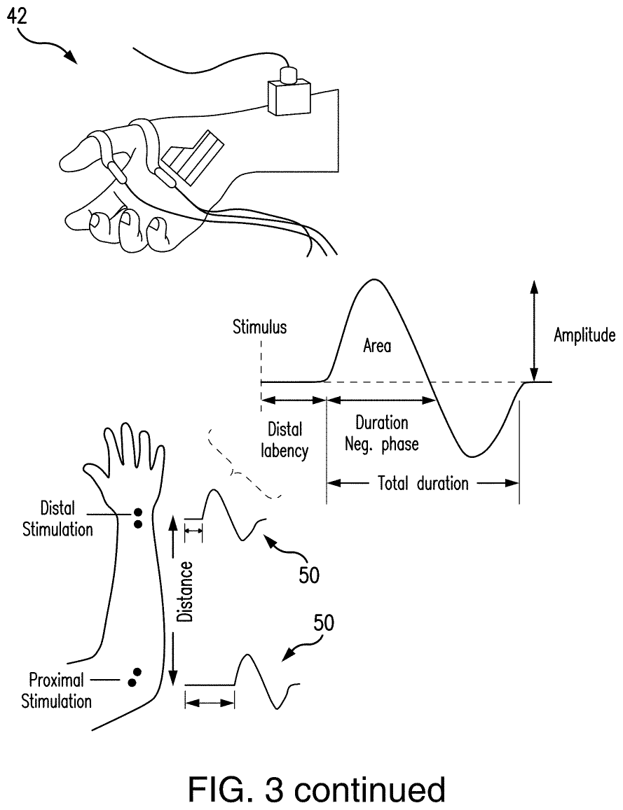

FIG. 3 shows an electromyography (EMG) measurement configurations 40, 42 in which a plurality of electrodes are used to collect muscular-related measurements from a subject. In the illustrated example, the leads generate a plurality of multi-channel time-synchronized signals 50. As will be discussed in greater detail, technology described herein converts the multi-channel time-synchronized signals 50 into a plurality of image patches, combines the image patches into an image, and generates, by a transformer neural network, a classification result based on the image. In one example, the classification result facilitates the automated detection of muscular disorders, anomalies and/or diseases (e.g., muscular dystrophy, polymyositis, etc.).

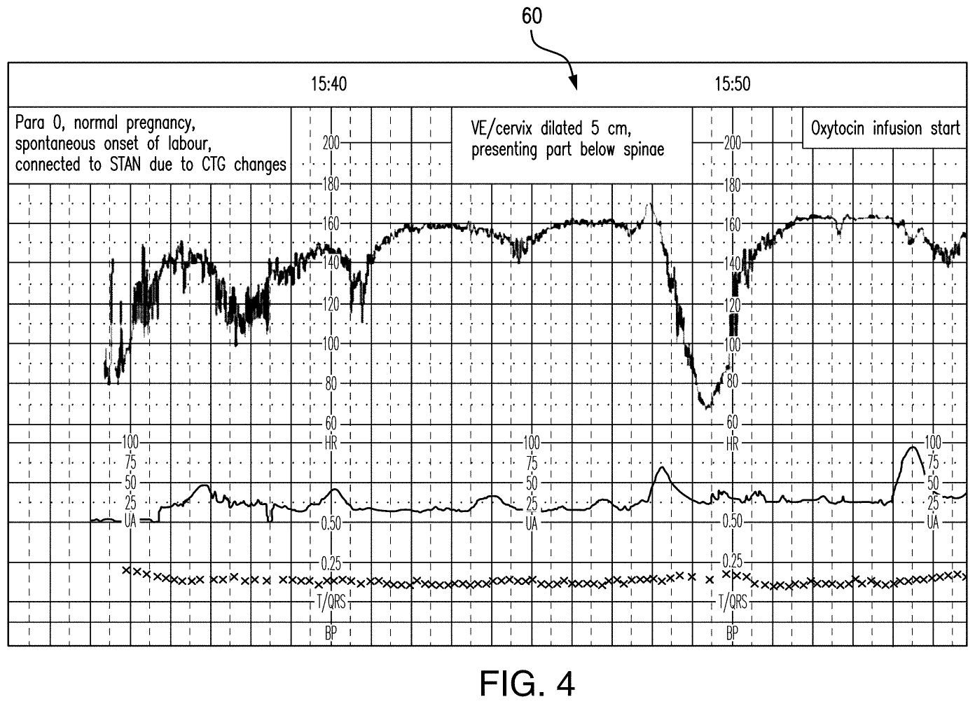

FIG. 4 shows a plurality of multi-channel time-synchronized signals 60 that are associated with a cardiotocography (CTG) measurement. As will be discussed in greater detail, technology described herein converts the multi-channel time-synchronized signals 60 into a plurality of image patches, combines the image patches into an image, and generates, by a transformer neural network, a classification result based on the image. In one example, the classification result facilitates the automated detection of maternal and/or fetal anomalies and/or diseases. The technology may also be used to analyze other types of signals such as, for example, ultrasound signals, radar signals, and so forth.

More particularly, embodiments provide a generalized deep learning solution for all signals based on a transformer neural network architecture. The signals are converted into a domain such that the signals can be fed into a transformer model. In one example, technology described herein handles the case of multi-channel time-synchronized signals, which are representative of real use cases in healthcare and other applications. Thus, although embodiments are discussed herein with respect to key challenges in healthcare (e.g., prediction of disease based on ECG signals, prediction of epilepsy based on intercranial EEG signals), other applications may also benefit from the technology described herein.

ECG

ECG is an established technology that is inexpensive and readily available. Therefore, ECG is a suitable use case for large scale training on non-standard data. Embodiments reached 91% accuracy in this area and may be extended to general signals.

Given a 12-lead ECG signal as an input (e.g., 12 separate signals recorded at 5,000 time steps), the ECG signal is converted into a 224 by 224 image. Fine tuning is then conducted using a Vision Transformer (ViT) model, which can be easily trained without tweaking of the model itself.

Some of the data may be lost, given that 12x5000 does not fit within 224x224, although the effect on the results is negligible. Embodiments can also use a model with a large input size, such as the 384×384 ViT model available in HUGGING FACE.

For the training data, over 100k anonymized samples of patients may be used, each labeled with a feature set or set of features such as gender, age, mortality, or any particular diseases of prediction interest.

The data may be split as:

50% training;

10% validation; and

40% hold-out test set.

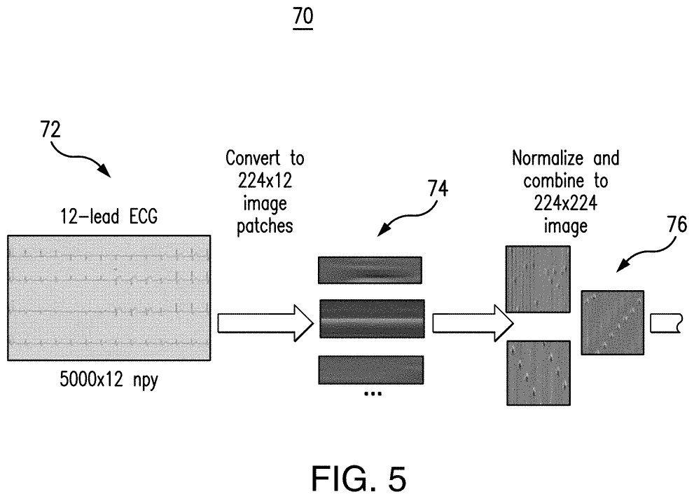

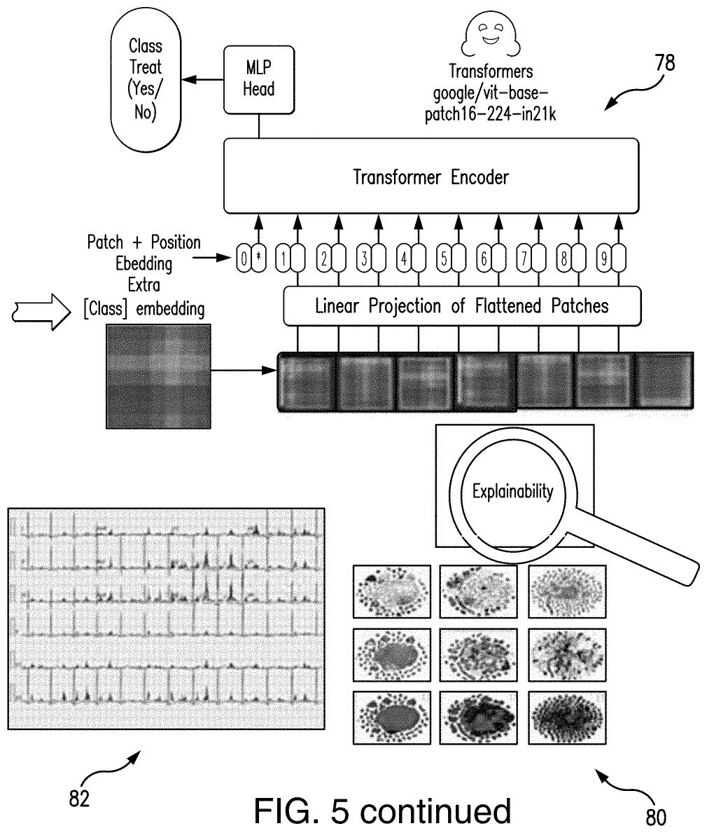

FIG. 5 shows an automated classification architecture 70 in which a plurality of multi-channel time-synchronized signals 72 (e.g., 12-lead ECG) are converted into a plurality of image patches 74. In the illustrated example, the image patches 74 are normalized and combined into an image 76, which is input to a transformer encoder 78 (e.g., transformer neural network) that generates a classification result 80. As will be discussed in greater detail, the classification result 80 may be transformed back into an original plot 82 for further analysis.

RESULTS

Table I shows that the results from the enhanced technology described herein surpass the accuracy of the state of the art (SOTA) AI solution by 7%, while training and inference runtime performance is 72% faster. Sensitivity and specificity are 16% and 13% higher, respectively, using the technology described herein. Results are based on the hold-out test set. such that data being tested was not included in the training set.

TABLE I

| | AUC | Sensitivity | Specificity |
|---|---|---|---|
| Gender (enhanced) | 0.97 | 91% | 93% |
| Gender (SOTA) | 0.90 | 75% | 88% |
| Mortality | 0.94 | 91% | 93% |
| (enhanced) | | | |

EXPLAINABILITY

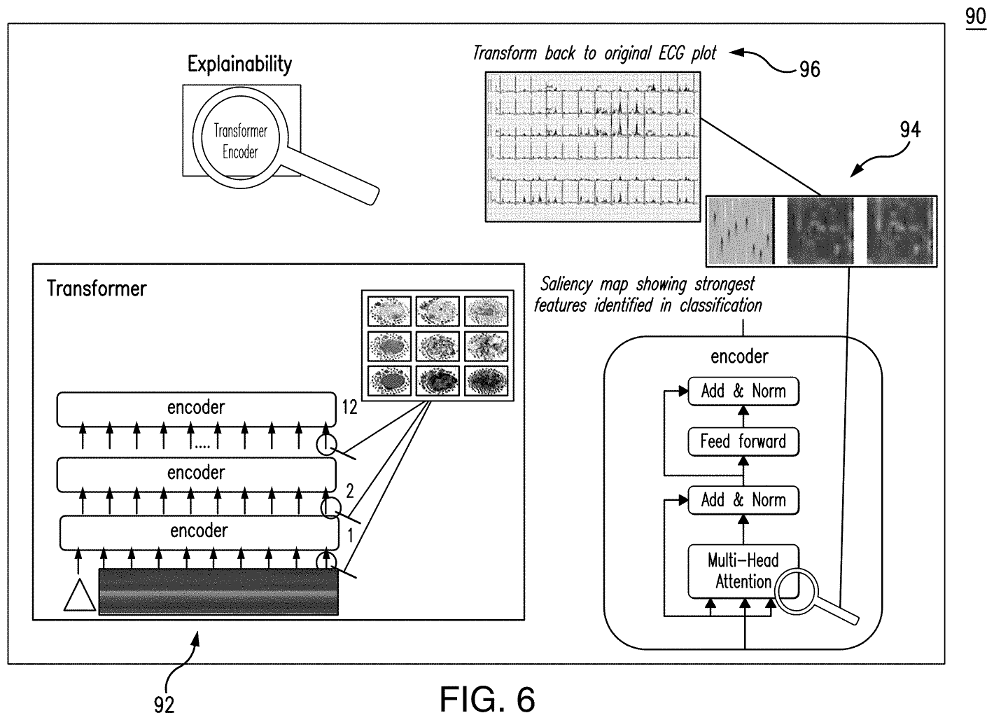

Turning now to FIG. 6, an advantage of using the technology described herein is the deep explainability 90 that can be gathered by observing the transformer throughout the various stages of the transformer. In one example, t-distributed stochastic neighbor embedding (T-SNE) information is gathered at each encoder stage 92, analyzing how the data is converging in classification. Embodiments are also able to observe a saliency map 94, find the strongest features that impacted the classification result, and transform back to the original signal to generate a human readable form 96 for the clinician.

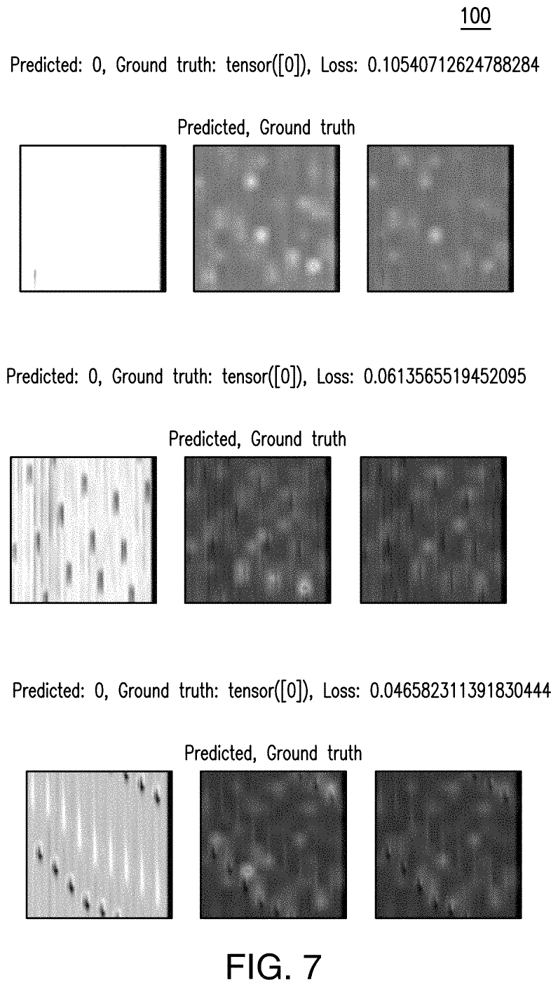

FIG. 7 shows a set of images 100 of a ground truth input (e.g., after transformation from signal to two-dimensional/2D space) and the representative per pixel explainability on the right. The images on the right can be projected back to the signal domain to show the saliency directly on the ECG signal. Each image is the transformed representation of an actual ECG signal, after being reordered and normalized according to highest and lowest peak in the signal, respectively.

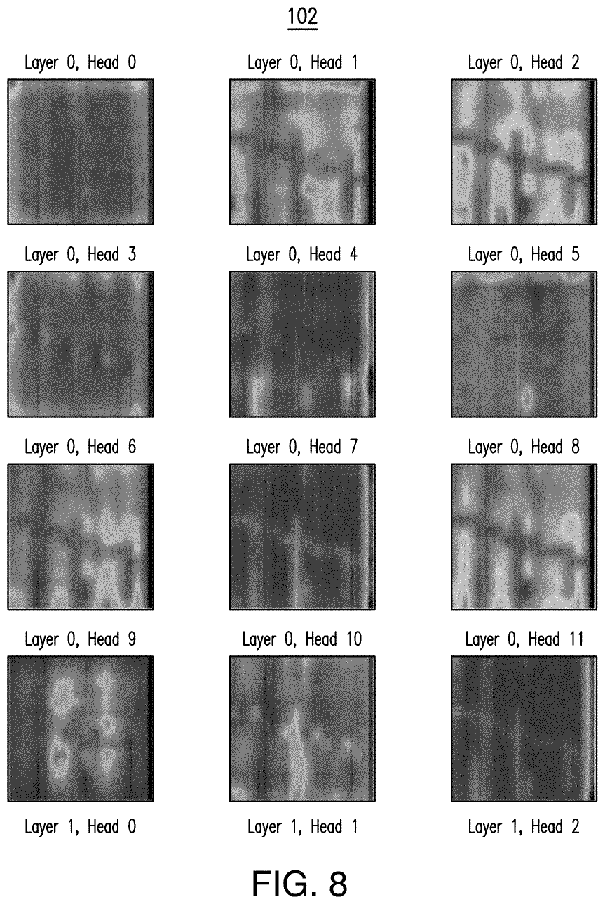

FIG. 8 shows a set of feature images 102 at each stage of the encoder layer. In the illustrated example, each head corresponds to a stage of the encoder layer. More particularly, the transformer architecture has a multiple encoder layer order in a hierarchical manner. The technology described herein observes each layer, extracts the temporary output of the layer, and performs a dimensionality reduction, which provides an indication of how/if the analysis is converging.

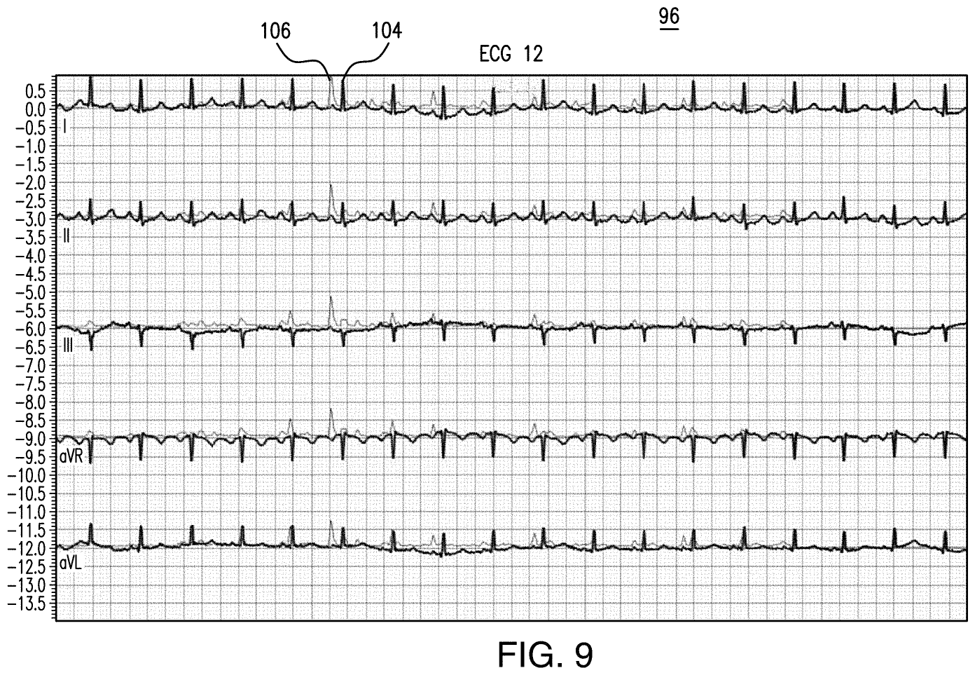

FIG. 9 demonstrates that the heatmap may be back-projected onto the original ECG plot in the human readable form 96 to determine which regions of the signal attributed most to the classification results. In the illustrated example, a first portion 104 shows the original ECG signal and a second portion 106 shows the strength of the attention.

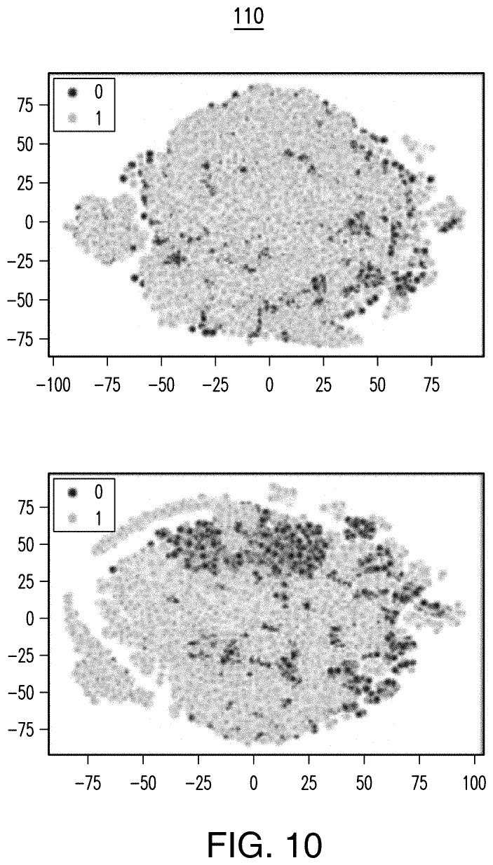

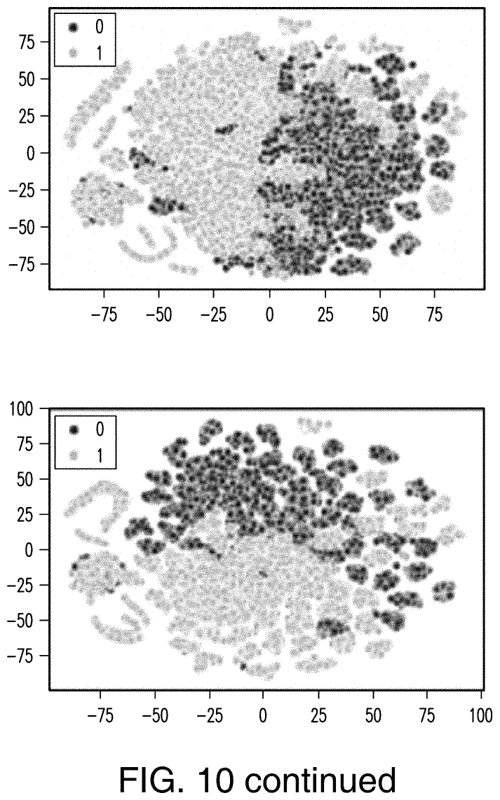

FIG. 10 shows a set of output T-SNE plots 110 to demonstrate how the data is classified as the token travels across the layers. The bottom image depicts the last layer, with a clear division between two classes.

EXTENDING TO FULL PRE-TRAINING

The technology described herein can be extended further by utilizing RGB (red, green, blue) channels instead of working with greyscale images (e.g., which set all RGB channels to the same value). This approach can be implemented by distributing the plurality of multi-channel time-synchronized signals across the set of RGB channels (e.g., placing a different signal channel in a separate RGB channel).

For the ECG example, with twelve channels per time-step:

Channel 1 in R, 2 in G, 3 in B of row r;

Channel 4 in R, 5 in G, 6 in B of row r+1;

Channel 7 in R, 8 in G, 9 in B of row r+2; and

Channel 10 in R, 11 in G, 12 in B of row r+3.

In this manner, the twelve channels are “compacted” into four rows only, instead of twelve. Therefore, since four is also a multiple of the 16×16 block-size, no padding is necessary.

Another benefit is that the total number of time samples that embodiments can support is larger than 5000. For example, floor(224/4)*224=12,544, which is larger than 5000, so in this case part of the image may be “wasted” (e.g., set to zeros or another constant value), but longer time recordings can be supported.

Another possible embodiment compacts channels into RGB separately. For example, in the current example of twelve channels, the channels will be compacted into 12/3=4 rows, each one RGB.

In this example, for acceptable performance of the transformer, the blocks are changed from 16×16 to 4xM, so that each block will hold information from a single local “time stamp” (only 4 rows together, not more). Moreover, if embodiments remain with 16 rows together in a single 16×16 block, then the technology described herein is arbitrarily bunching together information from four different time stamps, which is a multiple of 224 samples apart. In this scenario, remaining with 16×16 involves padding to 16 rows, to guarantee that separate time stamps remain separate.

In another example, embodiments can remain with 16×16 blocks without padding if 48 simultaneous channels are accommodated together (e.g., perhaps for EEG). Because 48/3=16, compacting into RGB separately will result in 16 rows corresponding to a single time stamp.

When using the RGB channels in a 224×224 image, with a transformer implementation using 16×16 blocks, then 16x3=48 different raw channels can be used simultaneously at each time instant. This approach results in the capability to record in a single 224×224 image the raw samples from floor(224/16)*224=14*224=3136 time instances.

EEG

Intracranial EEG (iEEG) data, is data recorded by multiple electrodes surgically implanted on the brain of a patient, unlike conventional EEG that records activity from outside the skull. Electrodes (typically 4-256) are either spread (e.g., grid) on the exposed cortex or deeply nested (depth) to record activity from deeper structures such as the hippocampus.

Since electrodes are implanted, data may be collected for hours and days to produce extremely large data sets for a relatively small (e.g., hundreds) number of patients. Data may be sampled at different rates to produce a nElectrodes* nSec* sample-rate data set.

Embodiments in this application may be evaluated using a subset of a publicly available dataset (e.g., Restoring Active Memory/RAM from University of Pennsylvania). A data sample, as defined by the domain expert, is a ten second (s) time window that maps into 5000 time points after resampling to 500 Hz (Hertz) and multiplied by the number of electrodes, which varies between patients. The samples may also be partially overlapping.

Two approaches (e.g., raw data and metrics) may be evaluated to apply the ViT model with this data.

Raw Data

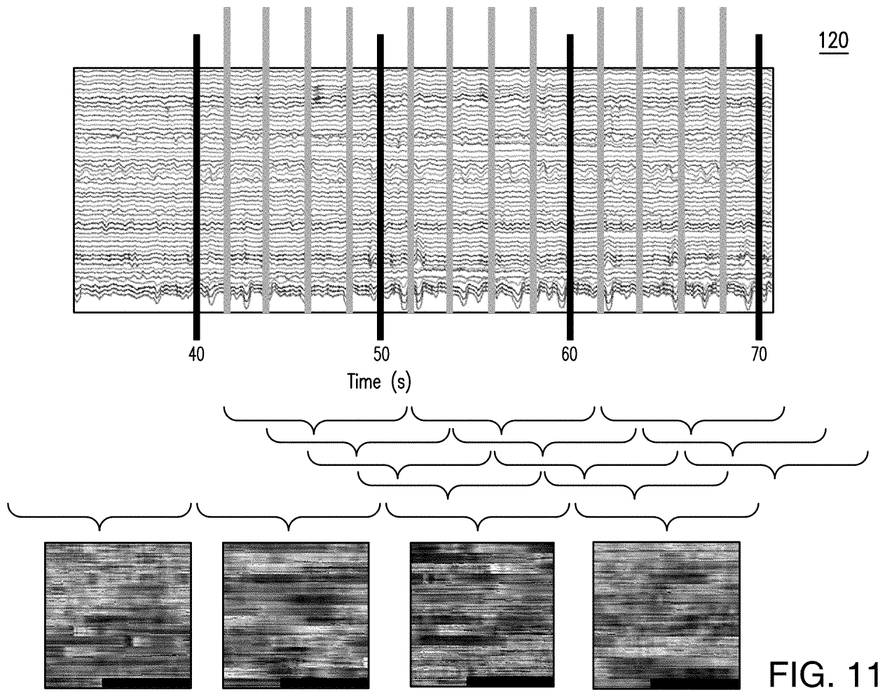

FIG. 11 shows a classification result 120 in which raw time samples are used as the input to the ViT model. For this approach, in order to conform to the 224x224×3 dimension and consider the 16×16×3 blocks units, embodiments may limit the number of electrodes and window size as:

nElectrodes*WindowSec*SampleRate<=3 *224^{∧}2

Note that the reduction is not the same across patients due to the different data shape (e.g., caused by a varying number of electrodes).

To comply with the above restriction, the recording from 48 electrodes may be used for the duration of six seconds (-6000 samples). Table II shows results for selected patients using this approach.

TABLE II

| | | 60 second windows - raw data | | | | | | |
|---|---|---|---|---|---|---|---|---|
| | | train/val/test = 80/10/10 | | | | | | |
| | | Number of | | | | | | |
| | | samples | | | | | | |
| | | (classified) | | | | | | |
| Patient | Band | 0, 1 | val_loss | val_accry | val_F1score | test_loss | test_accry | test_F1score |
| A | gamma | 11312, 2744 | 0.208 | 0.935 | 0.706 | 0.191 | 0.942 | 0.791 |
| B | gamma | 409, 4816 | 0.119 | 0.939 | 0.965 | 0.118 | 0.956 | 0.975 |
| C | gamma | 267, 8848 | 0.094 | 0.963 | 0.981 | 0.113 | 0.974 | 0.986 |
| D | gamma | 11938, 8162 | 0.276 | 0.704 | 0.68 | 0.666 | 0.725 | 0.697 |
| E | gamma | 2994, 7381 | 0.336 | 0.814 | 0.868 | 0.382 | 0.826 | 0.875 |
| F | gamma | 1074, 3788 | 0.317 | 0.82 | 0.873 | 0.381 | 0.778 | 0.834 |

An alternative is to tweak the ViT model. The advantage of this approach is that the ViT is exposed to the raw data and may discover hidden features.

A disadvantage may be the need for data reduction and the fact that the reduction is not the same across patients due to the different data shape (e.g., caused by a varying number of electrodes).

Metrics

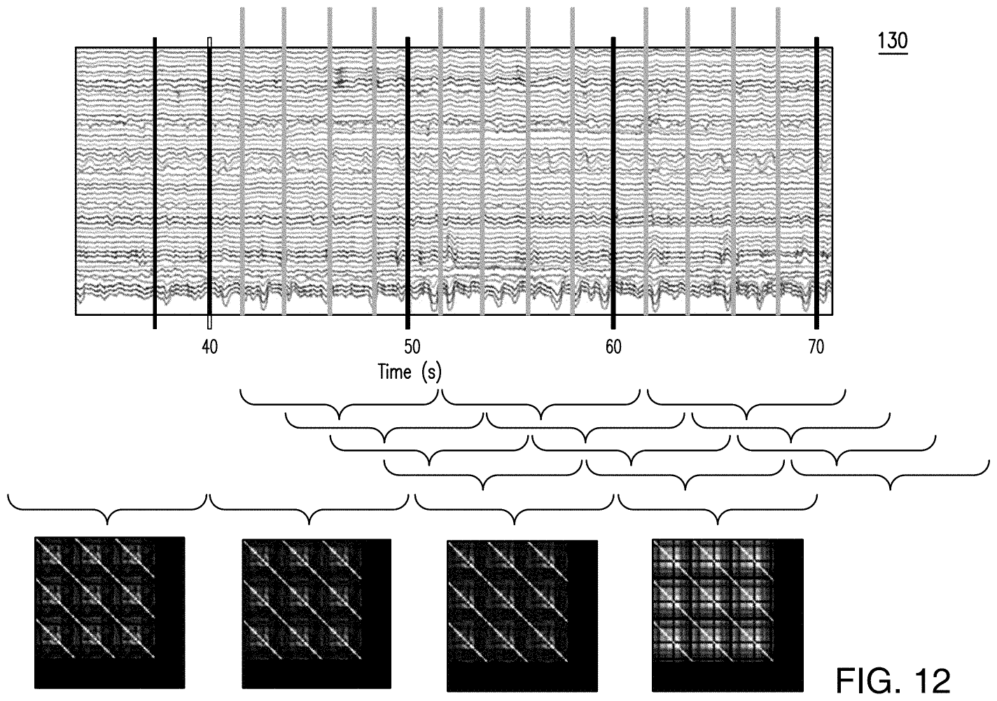

FIG. 12 shows a classification result 130 in which the relationship between the electrodes is relevant due to the nature of the use-case. Based on domain expertise, embodiments may choose a specific metric to use as feature and shape this metric as the ViT input matrix. Following this approach, a correlation matrix of nElectrodes*nElectrodes is created for each ten-second time window and a —0.9 prediction accuracy (f1 score) is reached. The same may be done with other metrics such as covariance, etc. The correlation matrix is padded—or may be duplicated and padded-to the complete 224×224 size. Table III shows results for selected patients using this approach.

TABLE III

| | | 10 second windows - correlation | | | | | | |
|---|---|---|---|---|---|---|---|---|
| | | train/val/test = 80/10/10 | | | | | | |
| | | Number of | | | | | | |
| | | samples | | | | | | |
| | | (classified) | | | | | | |
| Patient | Band | 0, 1 | val_loss | val_accry | val_F1score | test_loss | test_accry | test_F1score |
| A | gamma | | 0.265 | 0.888 | 0.785 | 0.278 | 0.896 | 0.782 |
| B | gamma | 110, 2730 | 0.094 | 0.986 | 0.993 | 0.072 | 0.964 | 0.981 |
| C | gamma | 27, 5437 | 0.008 | 0.996 | 0.998 | 0.03 | 0.994 | 0.997 |
| D | gamma | | 0.588 | 0.639 | 0.774 | 0.625 | 0.591 | 0.73 |
| E | gamma | 1118, 5097 | 0.161 | 0.861 | 0.915 | 0.323 | 0.866 | 0.919 |
| F | gamma | 347, 2567 | 0.108 | 0.904 | 0.943 | 0.249 | 0.886 | 0.929 |

An advantage of this approach is decoupling between data sample size and the ViT model matrix size. The approach can be applied to any data size as long as the recording channels number is <=224 without tweaking the model. For this specific use-case, where the number of electrodes is unlikely to be larger, the result is completely independent of the selected sample size.

Disadvantages are a priori feature selection and use-case dependent features (e.g., no generalization).

EXTENDING TO VIDEO TRANSFORMER

An alternate approach to deal with the large data size is to use a video transformer instead of a 2D transformer. In this way, the full raw data can be used without reduction, constructing a single matrix for each 1 second equivalent data so the limitation is: nElectrodes*SampleRate<3*2242. Assuming a 2-second overlap between time windows, every 50 matrices can be aggregated into a single 10-second (e.g., real time) video, which can be used to fine-tune a pre-trained video model.

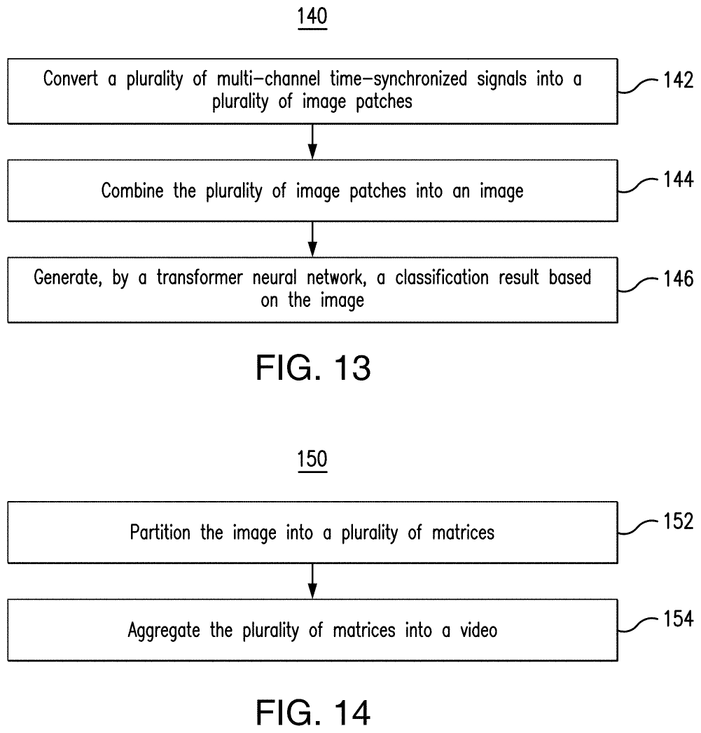

FIG. 13 shows a method 140 of classifying signals. The method 140 may be implemented in one or more modules as a set of logic instructions stored in a machine- or computer-readable storage medium such as random access memory (RAM), read only memory (ROM), programmable ROM (PROM), firmware, flash memory, etc., in hardware, or any combination thereof. For example, hardware implementations may include configurable logic, fixed-functionality logic, or any combination thereof. Examples of configurable logic (e.g., configurable hardware) include suitably configured programmable logic arrays (PLAs), field programmable gate arrays (FPGAs), complex programmable logic devices (CPLDs), and general purpose microprocessors. Examples of fixed-functionality logic (e.g., fixed-functionality hardware) include suitably configured application specific integrated circuits (ASICs), combinational logic circuits, and sequential logic circuits. The configurable or fixed-functionality logic can be implemented with complementary metal oxide semiconductor (CMOS) logic circuits, transistor-transistor logic (TTL) logic circuits, or other circuits.

Computer program code to carry out operations shown in the method 140 can be written in any combination of one or more programming languages, including an object oriented programming language such as JAVA, PYTHON, C++ or the like and conventional procedural programming languages, such as the “C” programming language or similar programming languages. Additionally, logic instructions might include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

Illustrated processing block 142 provides for converting a plurality of multi-channel time-synchronized signals into a plurality of image patches. In one example, the multi-channel time-synchronized signals are converted from a medical domain into the plurality of image patches. In such a case, the multi-channel time-synchronized signals might include one or more of ECG signals, EEG signals, EMG signals or CTG signals. Additionally, block 142 may distribute the plurality of multi-channel time-synchronized signals across a set of RGB channels (e.g., compacted). Block 144 combines the plurality of image patches into an image. In an embodiment, block 144 normalizes the image patches before the image patches are combined into the image. Block 146 generates, by a transformer neural network, a classification result based on the image. In one example, the transformer neural network is a 2D transformer neural network.

The method 140 therefore enhances performance at least to the extent that combining the plurality of multi-channel time-synchronized signals into an image preserves the key features of the signals while treating variance in a generalizable manner. Additionally, the use of the transformer neural network provides a simpler and more efficient solution that can be adapted to a wide variety of signals (e.g., without any convolutional neural network/CNN layers). Indeed, the transformer neural network may be a pre-trained transformer architecture for 2D images or three-dimensional (3D) video.

FIG. 14 shows a method 150 of using a video transformer neural network to classify signals. The method 150 may generally be incorporated into block 146 (FIG. 13), already discussed. More particularly, the method 150 may be implemented in one or more modules as a set of logic instructions stored in a machine- or computer-readable storage medium RAM, ROM, PROM, firmware, flash memory, etc., in hardware, or any combination thereof.

Illustrated processing block 152 provides for partitioning the image into a plurality of matrices. In an embodiment, block 154 aggregates the plurality of matrices into a video, wherein the video transformer neural network generates the classification result based on the video. The method 150 therefore further enhances performance by enabling the use of a pre-trained video transformer.

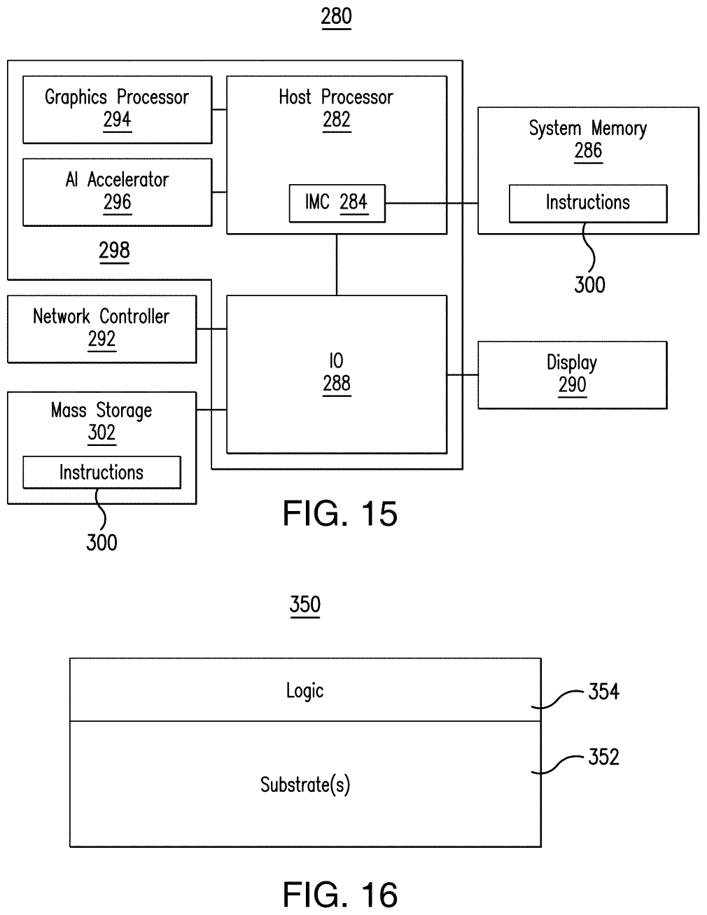

Turning now to FIG. 15, a performance-enhanced computing system 280 is shown. The system 280 may generally be part of an electronic device/platform having computing functionality (e.g., personal digital assistant/PDA, notebook computer, tablet computer, convertible tablet, server), communications functionality (e.g., smart phone), imaging functionality (e.g., camera, camcorder), media playing functionality (e.g., smart television/TV), wearable functionality (e.g., watch, eyewear, headwear, footwear, jewelry), vehicular functionality (e.g., car, truck, motorcycle), robotic functionality (e.g., autonomous robot), Internet of Things (IoT) functionality, etc., or any combination thereof

In the illustrated example, the system 280 includes a host processor 282 (e.g., central processing unit/CPU) having an integrated memory controller (IMC) 284 that is coupled to a system memory 286 (e.g., dual inline memory module/DIMM). In an embodiment, an IO (input/output) module 288 is coupled to the host processor 282. The illustrated IO module 288 communicates with, for example, a display 290 (e.g., touch screen, liquid crystal display/LCD, light emitting diode/LED display), mass storage 302 (e.g., hard disk drive/HDD, optical disc, solid state drive/SSD) and a network controller 292 (e.g., wired and/or wireless). The host processor 282 may be combined with the IO module 288, a graphics processor 294, and an AI accelerator 296 into a system on chip (SoC) 298.

In an embodiment, the host processor 282 and/or the AI accelerator 296 executes a set of program instructions 300 retrieved from the mass storage 302 and/or the system memory 286 to perform one or more aspects of the method 140 (FIG. 13) and/or the method 150 (FIG. 14), already discussed. Thus, execution of the illustrated instructions 300 by the host processor 282 and/or the AI accelerator 296 causes the host processor 282 and/or the AI accelerator 296 to convert a plurality of multi-channel time-synchronized signals into a plurality of image patches, combine the plurality of image patches into an image, and generate, by a transformer neural network, a classification result based on the image.

The instructions 300 may also be implemented in a distributed architecture (e.g., distributed in both location and over time). For example, the compacted encoding of raw signals into 2D images or 3D video may occur on a separate first processor (not shown) at an earlier time than the execution of the transformer-based neural network on the SoC 298 of the computing system 280 (e.g., a different separate remote second processor at a later time, independent of the earlier processing time). Furthermore, the results of a classification may be stored on a different separate remote third processor (not shown), to be displayed to a human user at a later time, independent of earlier processing times. Thus, the computing system 280 may be understood as illustrating one of a plurality of devices, rather than a single device.

Accordingly, the various processing stages may be initiated based on network messages between distributed processors, using suitable networking protocols, as known to those skilled in the art. For example, the TCP/IP (Transmission Control Protocol/Internet Protocol) suite of protocols, among others. The storage and retrieval of pre-processing, intermediate, and final results may be stored in databases using SQL (Structured Query Language) or No-SQL programming interfaces, among others. The storage elements may be physically located at different places than the processing elements.

The computing system 280 is therefore considered performance-enhanced at least to the extent that combining the plurality of multi-channel time-synchronized signals into an image preserves the key features of the signals while treating variance in a generalizable manner. Additionally, the use of the transformer neural network provides a simpler and more efficient solution that can be adapted to a wide variety of signals (e.g., without any CNN layers). Indeed, the transformer neural network may be a pre-trained transformer architecture for 2D images or 3D video.

FIG. 16 shows a semiconductor apparatus 350 (e.g., chip, die, package). The illustrated apparatus 350 includes one or more substrates 352 (e.g., silicon, sapphire, gallium arsenide) and logic 354 (e.g., transistor array and other integrated circuit/IC components) coupled to the substrate(s) 352. In an embodiment, the logic 354 implements one or more aspects of the method 140 (FIG. 13) and/or the method 150 (FIG. 14), already discussed.

The logic 354 may be implemented at least partly in configurable or fixed-functionality hardware. In one example, the logic 354 includes transistor channel regions that are positioned (e.g., embedded) within the substrate(s) 352. Thus, the interface between the logic 354 and the substrate(s) 352 may not be an abrupt junction. The logic 354 may also be considered to include an epitaxial layer that is grown on an initial wafer of the substrate(s) 352.

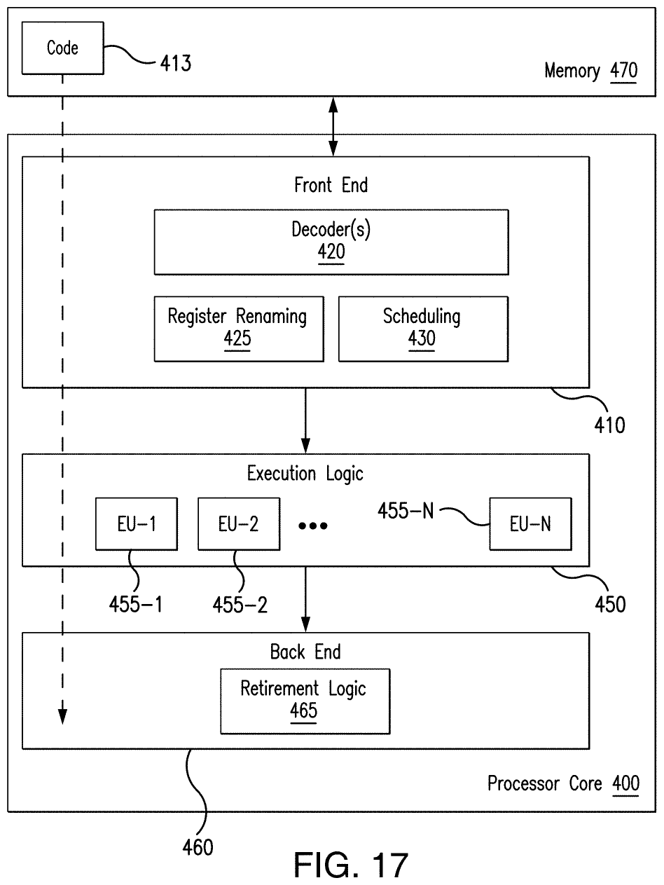

FIG. 17 illustrates a processor core 400 according to one embodiment. The processor core 400 may be the core for any type of processor, such as a micro-processor, an embedded processor, a digital signal processor (DSP), a network processor, or other device to execute code. Although only one processor core 400 is illustrated in FIG. 17, a processing element may alternatively include more than one of the processor core 400 illustrated in FIG. 17. The processor core 400 may be a single-threaded core or, for at least one embodiment, the processor core 400 may be multithreaded in that it may include more than one hardware thread context (or “logical processor”) per core.

FIG. 17 also illustrates a memory 470 coupled to the processor core 400. The memory 470 may be any of a wide variety of memories (including various layers of memory hierarchy) as are known or otherwise available to those of skill in the art. The memory 470 may include one or more code 413 instruction(s) to be executed by the processor core 400, wherein the code 413 may implement the method 140 (FIG. 13) and/or the method 150 (FIG. 14), already discussed. The processor core 400 follows a program sequence of instructions indicated by the code 413. Each instruction may enter a front end portion 410 and be processed by one or more decoders 420. The decoder 420 may generate as its output a micro operation such as a fixed width micro operation in a predefined format, or may generate other instructions, microinstructions, or control signals which reflect the original code instruction. The illustrated front end portion 410 also includes register renaming logic 425 and scheduling logic 430, which generally allocate resources and queue the operation corresponding to the convert instruction for execution.

The processor core 400 is shown including execution logic 450 having a set of execution units 455-1 through 455-N. Some embodiments may include a number of execution units dedicated to specific functions or sets of functions. Other embodiments may include only one execution unit or one execution unit that can perform a particular function. The illustrated execution logic 450 performs the operations specified by code instructions.

After completion of execution of the operations specified by the code instructions, back end logic 460 retires the instructions of the code 413. In one embodiment, the processor core 400 allows out of order execution but requires in order retirement of instructions. Retirement logic 465 may take a variety of forms as known to those of skill in the art (e.g., re-order buffers or the like). In this manner, the processor core 400 is transformed during execution of the code 413, at least in terms of the output generated by the decoder, the hardware registers and tables utilized by the register renaming logic 425, and any registers (not shown) modified by the execution logic 450.

Although not illustrated in FIG. 17, a processing element may include other elements on chip with the processor core 400. For example, a processing element may include memory control logic along with the processor core 400. The processing element may include I/O control logic and/or may include I/O control logic integrated with memory control logic. The processing element may also include one or more caches.

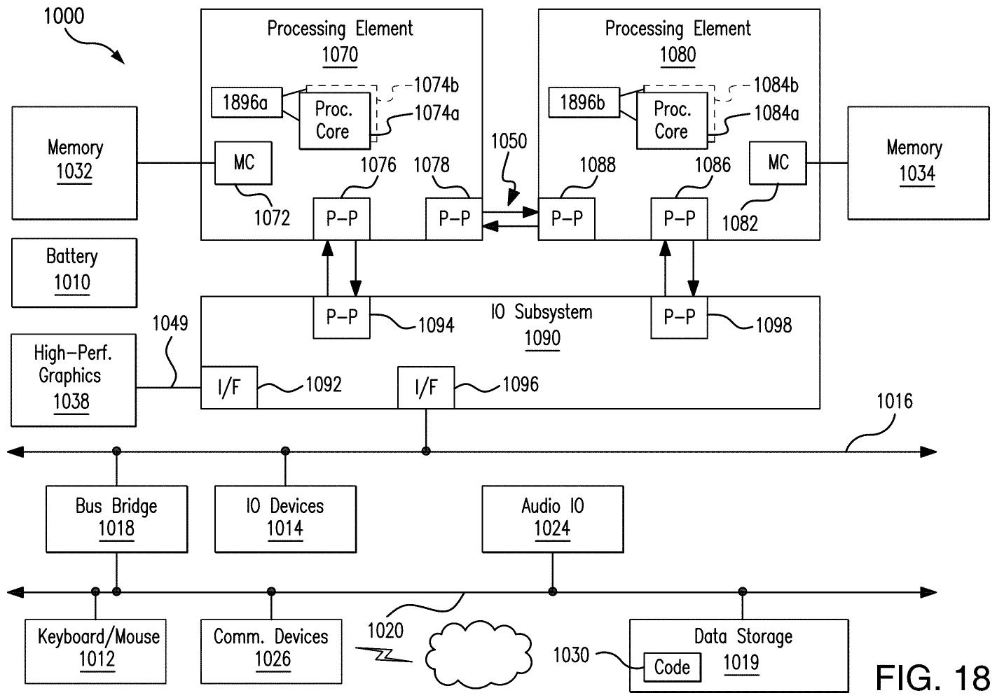

Referring now to FIG. 18, shown is a block diagram of a computing system 1000 embodiment in accordance with an embodiment. The computing system 1000 may be understood as illustrating one of a plurality of computer networks, rather than a single computer network. Shown in FIG. 18 is a multiprocessor system 1000 that includes a first processing element 1070 and a second processing element 1080. While two processing elements 1070 and 1080 are shown, it is to be understood that an embodiment of the system 1000 may also include only one such processing element.

The system 1000 is illustrated as a point-to-point interconnect system, wherein the first processing element 1070 and the second processing element 1080 are coupled via a point-to-point interconnect 1050. It should be understood that any or all of the interconnects illustrated in FIG. 18 may be implemented as a multi-drop bus rather than point-to-point interconnect.

As shown in FIG. 18, each of processing elements 1070 and 1080 may be multicore processors, including first and second processor cores (i.e., processor cores 1074a and 1074b and processor cores 1084a and 1084b). Such cores 1074a, 1074b, 1084a, 1084b may be configured to execute instruction code in a manner similar to that discussed above in connection with FIG. 17.

Each processing element 1070, 1080 may include at least one shared cache 1896a, 1896b. The shared cache 1896a, 1896b may store data (e.g., instructions) that are utilized by one or more components of the processor, such as the cores 1074a, 1074b and 1084a, 1084b, respectively. For example, the shared cache 1896a, 1896b may locally cache data stored in a memory 1032, 1034 for faster access by components of the processor. In one or more embodiments, the shared cache 1896a, 1896b may include one or more mid-level caches, such as level 2 (L2), level 3 (L3), level 4 (L4), or other levels of cache, a last level cache (LLC), and/or combinations thereof

While shown with only two processing elements 1070, 1080, it is to be understood that the scope of the embodiments are not so limited. In other embodiments, one or more additional processing elements may be present in a given processor. Alternatively, one or more of processing elements 1070, 1080 may be an element other than a processor, such as an accelerator or a field programmable gate array. For example, additional processing element(s) may include additional processors(s) that are the same as a first processor 1070, additional processor(s) that are heterogeneous or asymmetric to processor a first processor 1070, accelerators (such as, e.g., graphics accelerators or digital signal processing (DSP) units), field programmable gate arrays, or any other processing element. There can be a variety of differences between the processing elements 1070, 1080 in terms of a spectrum of metrics of merit including architectural, micro architectural, thermal, power consumption characteristics, and the like. These differences may effectively manifest themselves as asymmetry and heterogeneity amongst the processing elements 1070, 1080. For at least one embodiment, the various processing elements 1070, 1080 may reside in the same die package.

The first processing element 1070 may further include memory controller logic (MC) 1072 and point-to-point (P-P) interfaces 1076 and 1078. Similarly, the second processing element 1080 may include a MC 1082 and P-P interfaces 1086 and 1088. As shown in FIG. 18, MC's 1072 and 1082 couple the processors to respective memories, namely a memory 1032 and a memory 1034, which may be portions of main memory locally attached to the respective processors. While the MC 1072 and 1082 is illustrated as integrated into the processing elements 1070, 1080, for alternative embodiments the MC logic may be discrete logic outside the processing elements 1070, 1080 rather than integrated therein

The first processing element 1070 and the second processing element 1080 may be coupled to an I/O subsystem 1090 via P-P interconnects 1076 1086, respectively. As shown in FIG. 18, the I/O subsystem 1090 includes P-P interfaces 1094 and 1098. Furthermore, I/O subsystem 1090 includes an interface 1092 to couple I/O subsystem 1090 with a high performance graphics engine 1038. In one embodiment, bus 1049 may be used to couple the graphics engine 1038 to the I/O subsystem 1090. Alternately, a point-to-point interconnect may couple these components.

In turn, I/O subsystem 1090 may be coupled to a first bus 1016 via an interface 1096. In one embodiment, the first bus 1016 may be a Peripheral Component Interconnect (PCI) bus, or a bus such as a PCI Express bus or another third generation I/O interconnect bus, although the scope of the embodiments are not so limited.

As shown in FIG. 18, various I/O devices 1014 (e.g., biometric scanners, speakers, cameras, sensors) may be coupled to the first bus 1016, along with a bus bridge 1018 which may couple the first bus 1016 to a second bus 1020. In one embodiment, the second bus 1020 may be a low pin count (LPC) bus. Various devices may be coupled to the second bus 1020 including, for example, a keyboard/mouse 1012, communication device(s) 1026, and a data storage unit 1019 such as a disk drive or other mass storage device which may include code 1030, in one embodiment. The illustrated code 1030 may implement the method 140 (FIG. 13) and/or the method 150 (FIG. 14), already discussed. Further, an audio I/O 1024 may be coupled to second bus 1020 and a battery 1010 may supply power to the computing system 1000.

Note that other embodiments are contemplated. For example, instead of the point-to-point architecture of FIG. 18, a system may implement a multi-drop bus or another such communication topology. Also, the elements of FIG. 18 may alternatively be partitioned using more or fewer integrated chips than shown in FIG. 18.

Additional Notes and Examples:

Example 1 includes a performance-enhanced computing system comprising a network controller, a processor coupled to the network controller, and a memory coupled to the processor, the memory including a set of instructions, which when executed by the processor, cause the processor to convert a plurality of multi-channel time-synchronized signals into a plurality of image patches, combine the plurality of image patches into an image, and generate, by a transformer neural network, a classification result based on the image.

Example 2 includes the computing system of Example 1, wherein the plurality of multi-channel time-synchronized signals are converted from a medical domain into the plurality of image patches, and wherein the plurality of multi-channel time-synchronized signals are to include one or more of electrocardiogram signals, electroencephalogram signals, electromyography signals or cardiotocography signals.

Example 3 includes the computing system of Example 1, wherein the instructions, when executed, further cause the processor to distribute the plurality of multi-channel time-synchronized signals across a set of red, green and blue channels.

Example 4 includes the computing system of Example 1, wherein the instructions, when executed, further cause the processor to normalize the plurality of image patches before the plurality of image patches are combined into the image.

Example 5 includes the computing system of any one of Examples 1 to 4, wherein the transformer neural network is a two-dimensional transformer neural network.

Example 6 includes the computing system of any one of Examples 1 to 4, wherein the transformer neural network is a video transformer neural network, and wherein the instructions, when executed, further cause the processor to partition the image into a plurality of matrices, and aggregate the plurality of matrices into a video.

Example 7 includes at least one computer readable storage medium comprising a set of instructions, which when executed by a computing system, cause the computing system to convert a plurality of multi-channel time-synchronized signals into a plurality of image patches, combine the plurality of image patches into an image, and generate, by a transformer neural network, a classification result based on the image.

Example 8 includes the at least one computer readable storage medium of Example 7, wherein the plurality of multi-channel time-synchronized signals are converted from a medical domain into the plurality of image patches, and wherein the plurality of multi-channel time-synchronized signals are to include one or more of electrocardiogram signals, electroencephalogram signals, electromyography signals or cardiotocography signals.

Example 9 includes the at least one computer readable storage medium of Example 7, wherein the instructions, when executed, further cause the computing system to distribute the plurality of multi-channel time-synchronized signals across a set of red, green and blue channels.

Example 10 includes the at least one computer readable storage medium of Example 7, wherein the instructions, when executed, further cause the computing system to normalize the plurality of image patches before the plurality of image patches are combined into the image.

Example 11 includes the at least one computer readable storage medium of any one of Examples 7 to 10, wherein the transformer neural network is a two-dimensional transformer neural network.

Example 12 includes the at least one computer readable storage medium of any one of Examples 7 to 10, wherein the transformer neural network is a video transformer neural network, and wherein the instructions, when executed, further cause the computing system to partition the image into a plurality of matrices, and aggregate the plurality of matrices into a video.

Example 13 includes a semiconductor apparatus comprising one or more substrates, and logic coupled to the one or more substrates, wherein the logic is implemented at least partly in one or more of configurable or fixed-functionality hardware, the logic to convert a plurality of multi-channel time-synchronized signals into a plurality of image patches, combine the plurality of image patches into an image, and generate, by a transformer neural network, a classification result based on the image.

Example 14 includes the semiconductor apparatus of Example 13, wherein the plurality of multi-channel time-synchronized signals are converted from a medical domain into the plurality of image patches, and wherein the plurality of multi-channel time-synchronized signals are to include one or more of electrocardiogram signals, electroencephalogram signals, electromyography signals or cardiotocography signals.

Example 15 includes the semiconductor apparatus of Example 13, wherein the logic is further to distribute the plurality of multi-channel time-synchronized signals across a set of red, green and blue channels.

Example 16 includes the semiconductor apparatus of Example 13, wherein the logic is further to normalize the plurality of image patches before the plurality of image patches are combined into the image.

Example 17 includes the semiconductor apparatus of any one of Examples 13 to 16, wherein the transformer neural network is a two-dimensional transformer neural network.

Example 18 includes the semiconductor apparatus of any one of Examples 13 to 16, wherein the transformer neural network is a video transformer neural network, and wherein the logic is further to partition the image into a plurality of matrices, and aggregate the plurality of matrices into a video.

Example 19 includes the semiconductor apparatus of Example 13, wherein the logic coupled to the one or more substrates includes transistor channel regions that are positioned within the one or more substrates.

Example 20 includes a method of operating a performance-enhanced computing system, the method comprising converting a plurality of multi-channel time-synchronized signals into a plurality of image patches, combining the plurality of image patches into an image, and generating, by a transformer neural network, a classification result based on the image.

Example 21 includes the method of Example 20, wherein the plurality of multi-channel time-synchronized signals are converted from a medical domain into the plurality of image patches, and wherein the plurality of multi-channel time-synchronized signals include one or more of electrocardiogram signals, electroencephalogram signals, electromyography signals or cardiotocography signals.

Example 22 includes the method of Example 20, further including distributing the plurality of multi-channel time-synchronized signals across a set of red, green and blue channels.

Example 23 includes the method of Example 20, further including normalizing the plurality of image patches before the plurality of image patches are combined into the image.

Example 24 includes the method of any one of Examples 20 to 23, wherein the transformer neural network is a two-dimensional transformer neural network.

Example 25 includes the method of any one of Examples 20 to 23, wherein the transformer neural network is a video transformer neural network, and wherein the method further includes partitioning the image into a plurality of matrices, and aggregating the plurality of matrices into a video.

Example 26 includes an apparatus comprising means for performing the method of any one of Examples 20 to 25.

Technology described herein therefore enables AI (e.g., machine learning) tools to be created for medical practitioners (and perhaps also as basic building-blocks for start-ups in the medical domain). Moreover, the technology described herein may be used to keep staff up-to-date on new technology, and/or simply for positive public-relations.

Embodiments are applicable for use with all types of semiconductor integrated circuit (“IC”) chips. Examples of these IC chips include but are not limited to processors, controllers, chipset components, programmable logic arrays (PLAs), memory chips, network chips, systems on chip (SoCs), SSD/NAND controller ASICs, and the like. In addition, in some of the drawings, signal conductor lines are represented with lines. Some may be different, to indicate more constituent signal paths, have a number label, to indicate a number of constituent signal paths, and/or have arrows at one or more ends, to indicate primary information flow direction. This, however, should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments are not limited to the same. As manufacturing techniques (e.g., photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments. Further, arrangements may be shown in block diagram form in order to avoid obscuring embodiments, and also in view of the fact that specifics with respect to implementation of such block diagram arrangements are highly dependent upon the computing system within which the embodiment is to be implemented, i.e., such specifics should be well within purview of one skilled in the art. Where specific details (e.g., circuits) are set forth in order to describe example embodiments, it should be apparent to one skilled in the art that embodiments can be practiced without, or with variation of, these specific details. The description is thus to be regarded as illustrative instead of limiting.

The term “coupled” may be used herein to refer to any type of relationship, direct or indirect, between the components in question, and may apply to electrical, mechanical, fluid, optical, electromagnetic, electromechanical or other connections. In addition, the terms “first”, “second”, etc. may be used herein only to facilitate discussion, and carry no particular temporal or chronological significance unless otherwise indicated.

As used in this application and in the claims, a list of items joined by the term “one or more of may mean any combination of the listed terms. For example, the phrases “one or more of A, B or C” may mean A; B; C; A and B; A and C; B and C; or A, B and C.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

We claim:

1. A computing system comprising:
a network controller;
a processor coupled to the network controller; and
a memory coupled to the processor, the memory including a set of instructions, which when executed by the processor, cause the processor to:
convert a plurality of multi-channel time-synchronized signals into a plurality of image patches,
combine the plurality of image patches into an image, and
generate, by a transformer neural network, a classification result based on the image.

2. The computing system of claim 1, wherein the plurality of multi-channel time-synchronized signals are converted from a medical domain into the plurality of image patches, and wherein the plurality of multi-channel time-synchronized signals are to include one or more of electrocardiogram signals, electroencephalogram signals, electromyography signals or cardiotocography signals.

3. The computing system of claim 1, wherein the instructions, when executed, further cause the processor to distribute the plurality of multi-channel time-synchronized signals across a set of red, green and blue channels.

4. The computing system of claim 1, wherein the instructions, when executed, further cause the processor to normalize the plurality of image patches before the plurality of image patches are combined into the image.

5. The computing system of claim 1, wherein the transformer neural network is a two-dimensional transformer neural network.

6. The computing system of claim 1, wherein the transformer neural network is a video transformer neural network, and wherein the instructions, when executed, further cause the processor to:
partition the image into a plurality of matrices; and
aggregate the plurality of matrices into a video.

7. At least one computer readable storage medium comprising a set of instructions, which when executed by a computing system, cause the computing system to:
convert a plurality of multi-channel time-synchronized signals into a plurality of image patches;
combine the plurality of image patches into an image; and
generate, by a transformer neural network, a classification result based on the image.

8. The at least one computer readable storage medium of claim 7, wherein the plurality of multi-channel time-synchronized signals are converted from a medical domain into the plurality of image patches, and wherein the plurality of multi-channel time-synchronized signals are to include one or more of electrocardiogram signals, electroencephalogram signals, electromyography signals or cardiotocography signals.

9. The at least one computer readable storage medium of claim 7, wherein the instructions, when executed, further cause the computing system to distribute the plurality of multi-channel time-synchronized signals across a set of red, green and blue channels.

10. The at least one computer readable storage medium of claim 7, wherein the instructions, when executed, further cause the computing system to normalize the plurality of image patches before the plurality of image patches are combined into the image.

11. The at least one computer readable storage medium of claim 7, wherein the transformer neural network is a two-dimensional transformer neural network.

12. The at least one computer readable storage medium of claim 7, wherein the transformer neural network is a video transformer neural network, and wherein the instructions, when executed, further cause the computing system to:
partition the image into a plurality of matrices; and
aggregate the plurality of matrices into a video.

13. A semiconductor apparatus comprising:
one or more substrates; and
logic coupled to the one or more substrates, wherein the logic is implemented at least partly in one or more of configurable or fixed-functionality hardware, the logic to:
convert a plurality of multi-channel time-synchronized signals into a plurality of image patches;
combine the plurality of image patches into an image; and
generate, by a transformer neural network, a classification result based on the image.

14. The semiconductor apparatus of claim 13, wherein the plurality of multi-channel time-synchronized signals are converted from a medical domain into the plurality of image patches, and wherein the plurality of multi-channel time-synchronized signals are to include one or more of electrocardiogram signals, electroencephalogram signals, electromyography signals or cardiotocography signals.

15. The semiconductor apparatus of claim 13, wherein the logic is further to distribute the plurality of multi-channel time-synchronized signals across a set of red, green and blue channels.

16. The semiconductor apparatus of claim 13, wherein the logic is further to normalize the plurality of image patches before the plurality of image patches are combined into the image.

17. The semiconductor apparatus of claim 13, wherein the transformer neural network is a two-dimensional transformer neural network.

18. The semiconductor apparatus of claim 13, wherein the transformer neural network is a video transformer neural network, and wherein the logic is further to:
partition the image into a plurality of matrices; and
aggregate the plurality of matrices into a video.

19. The semiconductor apparatus of claim 13, wherein the logic coupled to the one or more substrates includes transistor channel regions that are positioned within the one or more substrates.

20. A method comprising:
converting a plurality of multi-channel time-synchronized signals into a plurality of image patches;
combining the plurality of image patches into an image; and
generating, by a transformer neural network, a classification result based on the image.

21. The method of claim 20, wherein the plurality of multi-channel time-synchronized signals are converted from a medical domain into the plurality of image patches, and wherein the plurality of multi-channel time-synchronized signals include one or more of electrocardiogram signals, electroencephalogram signals, electromyography signals or cardiotocography signals.

22. The method of claim 20, further including distributing the plurality of multi-channel time-synchronized signals across a set of red, green and blue channels.

23. The method of claim 20, further including normalizing the plurality of image patches before the plurality of image patches are combined into the image.

24. The method of claim 20, wherein the transformer neural network is a two-dimensional transformer neural network.

25. The method of claim 20, wherein the transformer neural network is a video transformer neural network, and wherein the method further includes:
partitioning the image into a plurality of matrices; and
aggregating the plurality of matrices into a video.
